(19) United States
(12) Patent Application Publication
(10) Pub. No.: US 2024/0028790 A1
(43) Pub. Date: Jan. 25, 2024

(54) METHODS AND SYSTEMS FOR SELECTING CONDITIONS FOR MAKING INHALATION FORMULATIONS

(71) Applicants: AMGEN INC., Thousand Oaks, CA (US); Board of Regents, The University of Texas System, Austin, TX (US)

(72) Inventors: Tian Wu, Newbury Park, CA (US); Michael Schneider, Thousand Oaks, CA (US); Ryan Khalaf, Thousand Oaks, CA (US); Hugh Smyth, Austin, TX (US); Ashlee Brunaugh, Austin, TX (US); Li Ding, Austin, TX (US)

(21) Appl. No.: 18/256,442

(22) PCT Filed: Dec. 22, 2021
(86) PCT No.: PCT/US21/64773

§ 371 Date: Jun. 8, 2023

Related U.S. Application Data
(60) Provisional application No. 63/130,063, filed on Dec. 23, 2020.

(51) Int. Cl. G06F 30/20

(52) U.S. Cl. CPC G06F 30/20

(57) ABSTRACT

A forecasting modeling computing system includes a processors and a memory including a set of computer-executable instructions that, when executed by the processor, cause the forecasting modeling computing system to receive design parameters, determine a predicted median particle size, identify a predictive quadratic model, and display a response surface visualization. A computer-implemented method includes receiving design parameters, determining a predicted median particle size, identifying a predictive quadratic model; and display a response surface visualization.

### CROSS-REFERENCE TO RELATED APPLICATIONS

This application claims priority to and the benefit of U.S. Provisional Application No. 63/130,063, filed on Dec. 23, 2020, entitled “Methods and Systems for Selecting Conditions for Making Inhalation Formulations,” the entire disclosure of which is hereby expressly incorporated by reference herein.

### FIELD OF THE INVENTION

The present disclosure is generally directed to the field of predictive modeling methods and systems for selecting optimal spray drying process conditions in particle engineering, and more particularly, to predictive modeling techniques for selecting optimal spray drying process conditions to generate stable and inhalable protein particles for dry powder inhalation formulations.

### BACKGROUND

A burgeoning therapeutic market now exists for protein formulations. For example, monoclonal antibodies (mAbs) are being developed and brought to market rapidly, with 18 new antibodies approved by the Food and Drug Administration (FDA) between 2018 and 2019. However, administration of mAbs is currently limited to injection (e.g., subcutaneous or intravenous) routes, and no FDA-approved inhalable mAb products exist. This state of affairs is largely due to the fact that existing particle engineering techniques have failed to address practical aspects necessary to the development and deployment of inhalable protein therapeutics, such as mAbs.

Inhalable mAb powder requires both suitable processing and delivery aspects for commercial viability. Yet the complexity and lability of mAbs represent a substantial barrier to alternative routes of administration including inhalation, as evidenced by the failure of conventional techniques to develop inhalable mAbs that achieve both aerosol performance and protein stability.

For example, conventional efforts have included analyzing powder properties relevant to protein stability (e.g., crystallinity) of respirable anti-Immunoglobulin E (IgE) mAb powders produced using spray drying and spray freeze drying. However, such techniques have failed to address the effect of processing and formulation conditions on the mAb structure and formation of aggregates. Other efforts have evaluated the stability of spray-dried Immunoglobulin G1 (IgG1) in combination with varying levels of mannitol and determined that a mannitol content of at least 20-30% resulted in stabilizing capacity. However, generated formulations exhibited low aerosol performance.

Pulmonary delivery of protein therapeutics, such as mAbs, is an attractive option for targeted treatment of lung diseases. To enter the deep airways of the lungs, droplets or particles (e.g., such as those emitted from a delivery device) require an aerodynamic diameter of less than 3-5 μm. However, to generate particles (herein, “droplets” are understood to refer to particles) having a suitable aerodynamic diameter such that the droplet can reach the depths of the lungs, the atomization used in conventional particle engineering techniques requires the use of stress-inducing processes. Such stress-inducing processes cause, inter alia, structural changes to the protein composing the droplets, binding/aggregation of the droplets, loss of therapeutic activity, and/or an immunogenic response.

### SUMMARY

In one aspect, a forecasting computing system for optimizing atomization settings during particle engineering of a protein includes one or more processors and a memory including a set of computer-executable instructions. The instructions, when executed by the one or more processors, may cause the forecasting computing system to receive, in a design generation module of the memory, a user selection of a plurality of design parameters with respect to a statistical design. The memory may include further instructions that, when executed, cause the forecasting computing system to determine, in a suitability assessment module of the memory, a predicted median particle size; and identify, in a stability assessment module of the memory, one or more predictive quadratic models by fitting each of one or more response variables assessed in a statistical experiment corresponding to the statistical design. The memory may include further instructions that, when executed, cause, in a visualization module of the memory, for each of the one or more predictive quadratic models, a response surface visualization to be displayed in a display device of a user.

In another embodiment, a computer-implemented method for determining optimal formulation atomization settings in a particle engineering process of a protein includes receiving, in a design generation module of a forecasting computing system, a user selection of a plurality of design parameters with respect to a statistical design; determining, in a suitability assessment module of the forecasting computing system, a predicted median particle size; identifying one or more predictive quadratic models by fitting each of one or more response variables assessed in a statistical experiment corresponding to the statistical design; and causing, in a visualization module of the forecasting computing system, for each of the one or more predictive quadratic models, a response surface visualization to be displayed in a display device of a user.

### BRIEF DESCRIPTION OF THE FIGURES

FIG. 1 depicts an exemplary forecasting computing environment for performing predictive modeling techniques to select optimal spray drying process conditions to generate stable and inhalable protein particles for dry powder inhalation formulations, according to one embodiment.

FIG. 2A depicts an exemplary particle processing method, according to one embodiment and scenario.

FIG. 2B depicts an exemplary a stored particle analysis method for performing stability assessment, according to one embodiment and scenario.

FIG. 3A depicts an exemplary response surface visualization of predicted median particle diameter, according to one embodiment and scenario.

FIG. 3B depicts exemplary response surface visualizations of increase in beta shoot of increase in relative area of dimer species, according to one embodiment and scenario.

FIG. 3C depicts exemplary response surface visualizations of increase in beat shoot content given varying solid content, atomization rate and feed rate parameters, according to one embodiment and scenario.

FIG. 3D depicts an exemplary response surface visualization of increase in beta turns content, according to one embodiment and scenario.

FIG. 3E depicts an exemplary response surface visualization of decrease in unordered content, according to one embodiment and scenario.

FIG. 4 depicts an exemplary method for determining optimal formulation atomization settings in a particle engineering process of a protein.

### DETAILED DESCRIPTION

The embodiments described herein relate to, inter alia, techniques for selecting optimal spray drying process conditions in particle engineering, and more particularly, to predictive modeling techniques for selecting optimal spray drying process conditions to generate stable and inhalable protein (e.g., antibody, such monoclonal antibody (mAb)) particles for dry powder inhalation formulations. The present techniques further include methods and systems for particle processing and stored particle analysis.

Direct deposition of a mAb in the airways has been experimentally determined to result in prolonged drug concentrations in the lung with low and slow passage into the blood stream. The targeted nature of the pharmacokinetic profile of inhalable mAbs is thought to be beneficial in treating both chronic lung diseases (e.g., asthma, chronic obstructive pulmonary disease, lung cancer, etc.) as well as diseases that provoke acute infections and/or inflammatory responses, particularly in the case of SARS-CoV-2, for which antibody-based therapies are a promising approach.

However, as noted above, despite many years of research and development devoted to developing therapeutic products including mAbs, efficient production of inhalable mAbs on an industrial scale remains a challenge because such production requires a full understanding not only of the action of the mAb but also of the pathways and mechanisms of degradation related to processing, storage, and delivery of the mAb.

The present techniques advantageously provide techniques for generating formulations of biopharmaceuticals in a dried, solid state, which includes stabilizing advantages compared to storage in the liquid state. The ability to produce stable powdered mAbs results in several practical benefits. First, for inhalation-based delivery, the present techniques may include reconstituting a product generated using the present techniques (e.g., a lyophilized cake) in a solution. The present techniques may include delivering the solution via a delivery system (e.g., a nebulizer). In another embodiment, the present techniques may include loading a formulation of protein (e.g., antibodies, such as mAbs) as a respirable powder in a dry powder inhaler (DPI). Delivery of antibodies (e.g., mAbs) via DPI is particularly advantageous. Compared to nebulizers, DPIs offer greater portability, require no external power supply, and have shorter administration times. Therefore, the present techniques advantageously improve patient compliance and simplify dosage.

The present techniques may be used in conjunction with both spray drying and spray freeze drying techniques, and may include comparing the robustness of respective powders produced using the particle engineering techniques of spray drying and spray freezing drying. The comparing may include performing stress testing in storage conditions typical for a DPI product. It will be appreciated by those of ordinary skill in the art that respective powders produced using spray drying and spray freeze drying techniques may have contrasting physiochemical properties, leading to differing long-term protein stability, especially for labile proteins such as mAbs. As such, spray drying may be a preferred technique in some applications.

The present techniques include determining appropriate processing and formulation conditions for delivery of a variety of therapeutic proteins, including but not limited to mAbs, by examining each aspect of the particle engineering process (atomization, freezing and drying) for instability. In some embodiments, the present techniques may compare instability generated using the present techniques to baseline instability produced in lyophilization, a technique conventionally used for the production of solid-state mAb pharmaceuticals. In some embodiments and scenarios, anti-streptividin IgG1 may be used as a model protein. However, those of ordinary skill in the art will appreciate that the present techniques apply to the development of other protein therapeutics, including but not limited to inhalable vaccines, other therapeutic antibodies, etc.

The invention is further described in the following examples. The example serves only to illustrate the invention and is not intended to limit the scope of the invention in any way.

Exemplary Forecasting Computing Environment

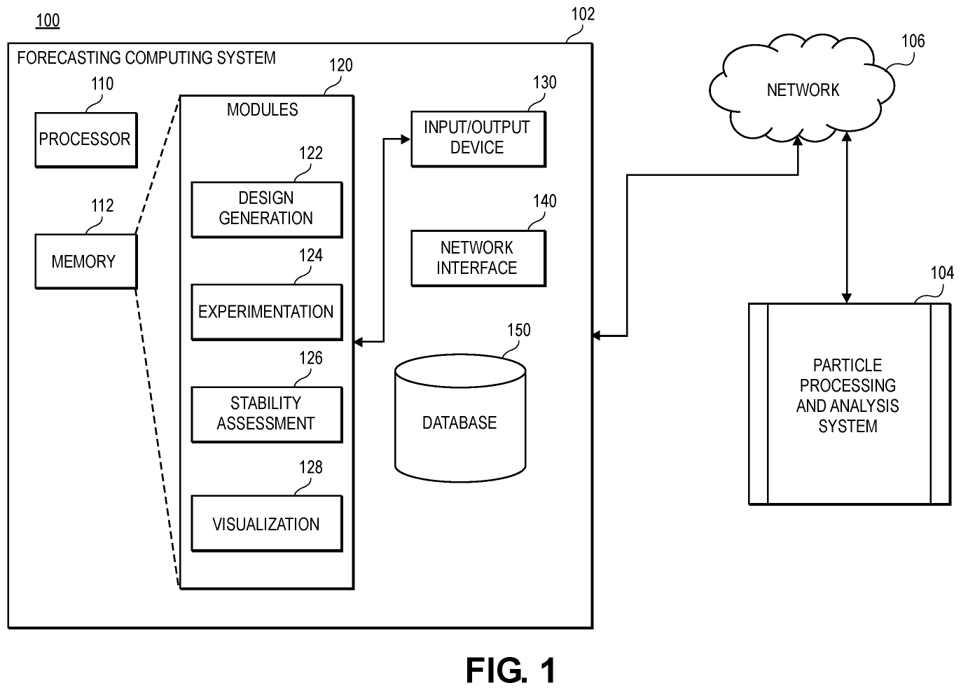

Turning to FIG. 1, an exemplary forecasting modeling computing environment 100 for performing predictive modeling techniques to select optimal spray drying process conditions to generate stable and inhalable protein particles for dry powder inhalation formulations is depicted. The computing environment 100 includes a forecasting computing system 102 that may comprise one or more computers that may be respectively implemented in a virtualized and/or cloud computing environment, for example. The computing environment 100 further includes a particle processing and analysis system 104 and a network 106 communicatively coupling the forecasting computing system 102 and the particle processing and analysis system 104. For example, and without limitation, the network 106 may include one or more suitable wireless networks, such as a 3G or 4G network, a WiFi network or other wireless local area network (WLAN), a satellite communication network, and/or a terrestrial microwave network, for example. In some embodiments, the network 106 also includes one or more wired networks, such as Ethernet.

The forecasting computing system 102 may include a processor 110 and a memory 112. While referred to in the singular, the processor 110 may include any suitable number of processors of one or more types (e.g., one or more central processing units (CPUs), graphics processing units (GPUs), cores, etc.). The memory 112 may comprise one or more memories of one or more types (e.g., persistent memory, solid state memory, random access memory (RAM), etc.), and may store one or more modules 120. The one or more modules 114 may include, for example, a design generation module 122, an experimentation module 124, a stability assessment module 126 and a visualization module 128.

The design generation module 122 may include computer-executable instructions that, when executed by the processor 110, are capable of generating, modifying and executing one or more statistical designs (i.e., design of experiments, or “DoE”), including but not limited to factorial designs (e.g., general full-factorial designs, two-level full factorial designs, Plackett-Burman designs, etc.), response-surface designs (e.g., Box-Behnken designs, central composite designs, etc.) and/or randomized designs (e.g., Latin-hypercube designs). The computer-executable instructions comprising the design generation module 122 may be authored in one or more suitable programming language (e.g., C, C++, R, Java, Python, JavaScript, LISP, etc.) and may include algorithms for performing the enumerated statistical designs, as well as additional instructions for assisting end users (e.g., engineers, scientists, programmers, quality assurance testers, statisticians, etc.) in constructing, parameterizing, evaluating and/or interpreting (e.g., by visualizing) experimental designs and the outputs/results thereof, via one or more input/output device.

In some embodiments, specialized packages available for use in conjunction with the suitable programming language(s) may be included in, or accessible to, the design generation module 122, such as the rsm package in R for fitting linear models with a response-surface component. The design generation module 112 may include one or more runtimes for executing configured (i.e., parameterized) statistical designs, and/or yet further computer-executable instructions for storing the parameterized designs, and/or results of the one or more statistical designs, in the memory 112 or in another location (e.g., in an electronic database). The configuration and operation of the design generation module 122 are discussed in further detail below.

In some embodiments, a computer application (not depicted) may provide a wrapper, or package, for some of the functionality associated with one or more of the modules 120. In particular, the application may contain instructions that, when executed at a device, enable the device to use one or more of the modules 120. Specifically, after a user configures one or more statistical designs, the application may package and make available for download to one or more (potentially very many) other devices, including mobile computing devices. The application may comprise mobile application programming instructions and/or software (e.g., an Android Package Kit (APK) file), for example.

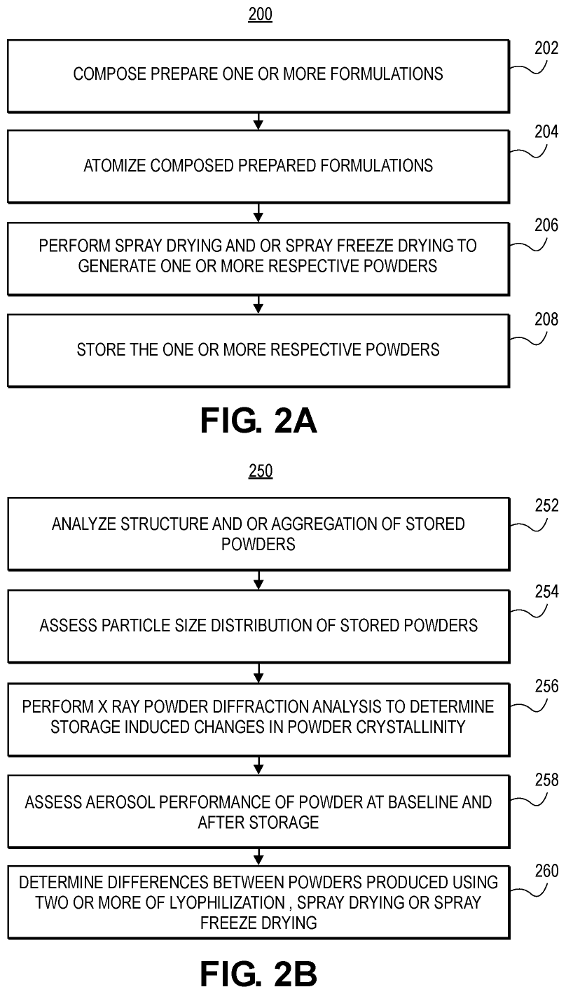

The experimentation module 124 may include computer-executable instructions that, when executed, perform one or more experimental functions. The experimental functions may range from fully automated experimental steps (e.g., an automated particle processing method) to receiving data from a manual experimental procedure performed by a user (e.g., a lab technician). The experimentation module 124 may include computer-executable instructions for receiving/retrieving information from the particle processing and analysis system 104, such as experimental metadata (e.g., an experimental series, a protein identifier, etc.). The experimentation module 124 may include instructions for accessing data in the particle processing and analysis system 104, such as the current status of experimental variables, and for controlling the particle processing and analysis system 104. For example, the experimentation module 124 may include instructions for causing specialized hardware or software functions of the particle processing and analysis system 104 (e.g., an x-ray powder diffraction (XRD) analysis) to be actuated. In general, the experimentation module 124 includes computer-executable instructions for performing one or more of the steps of the particle processing method 200 depicted in FIG. 2A and/or one or more of the steps of the stored particle analysis method 250 depicted in FIG. 2B.

The stability assessment module 126 includes computer-executable instructions for assessing the stability of a target protein under varying atomization conditions, according to a plurality of response variables determined by the design generation module 122 and using experimental data provided by the experimentation module 124. For example, the stability assessment module may include instructions for predicting a median particle size according to an equation:

$${D}_{d} = \frac{{D}_{w}}{{( \frac{{ρ}_{D}}{{ρ}_{w}} \cdot \frac{{C}_{D}}{{C}_{w}} )}^{\frac{1}{3}}}$$

where D_{d} is the predicted median particle diameter, D_{w} is the median droplet diameter (obtained, for example, by the particle processing and analysis system 104 using laser diffraction analysis of the spray), ρ_{D} is the density of the particle, ρ_{W} is the density of the droplet, C_{w} is the solid fraction of the droplet, and C_{D} is the solid fraction of the droplet.

Specifically, the stability assessment module 126 may use this equation in some embodiments to predict the suitability of the atomization conditions assessed in an atomization statistical model (e.g., a Box-Behnken DoE model) in producing respirable spray dried particles and/or respirable spray freeze dried particles.

In some embodiments, the stability assessment module 126 includes instructions for fitting one or more quadratic models to each separate response variable of an atomization statistical model by considering increases and increases in the response variables for the protein relating to the atomization statistical model as opposed to that of the unprocessed protein. The stability assessment module 126 may include instructions for considering all decreases in a particular factor as equivalent to zero in cases where only increases were considered, and/or for considering all increases as equivalent to zero in cases where only decreases in a factor were considered. The stability assessment module 126 may include instructions for considering all independent variables of the statistical model and for assessing a relationship between both the average response of a number (e.g., 15) of unprocessed samples as well as a pair-wise comparison for unprocessed and processed samples for a given run. The stability assessment module 126 may include further instructions for removing insignificant terms from the model.

The stability assessment module 126 may include further instructions for determining, based on an F-test and lack of fit test, whether a given statistical model is appropriate for predictive purposes. For example, the stability assessment module 126 may include instructions requiring that the F-test be significant (i.e., P<0.1) and/or that the lack of fit test be insignificant (i.e., P>0.1). Moreover, the stability assessment module 126 may, for those models deemed suitable for predictive purposes, optimize the models using a desirability function to determine the most appropriate atomization settings to achieve a respirable particle with limited effects on protein stability:

$$d \frac{min}{r} \{ \begin{matrix}0 & if {f}_{r} ( x ) > B \\ ( \frac{{f}_{r} ( x ) - {B}^{s}}{A - B} ) & if A \leq {f}_{r} ( x ) \leq B \\ 1 & {f}_{r} ( x ) < A\end{matrix} \begin{matrix} \\ \end{matrix}$$

where

$$d \frac{min}{r}$$

is we desirability function winch is set to minimize the response (i.e., median predicted particle size and changes in protein stability), fᵣ(x) is the function to optimize (i.e., the predictive quadratic model(s)), s is an adjustable scaling factor, and A and B represent, respectively, the upper and lower limits of the response (e.g., the standard deviation of the unprocessed samples for the stability response variable. In some embodiments, an existing software package (e.g., R desirability) may be used to implement the desirability optimization.

The stability assessment module 126 may include instructions for generating one or more predictive quadratic equations corresponding to, for example, a predicted particle diameter, an increase in SEC-HPLC HMW peak area vs. average unprocessed control, an increase in beta strand content versus unprocessed pair-wise control, an increase in beta turns content versus unprocessed pair-wise control, a decrease in unordered content versus unprocessed pair-wise control. The one or more predictive quadratic equations may be visualized (e.g., in a response surface) via the visualization module 128.

The visualization module 128 may generate one or more visualization representing the output of the stability assessment module 126. For example, the visualization module 128 may include computer-executable instructions for generating one or more response surface corresponding to each quadratic equation, wherein the response surface includes indications of which response variables play an influential role in the predicted particle size and protein structural changes post-atomization. Visualizations are discussed further below. The visualization module 128 may also include instructions for causing one or more visualizations to be generated and displayed in an I/O device (e.g., an I/O device of the modeling computing system 102, a mobile device of a user, etc.).

The forecasting computing system 102 may also comprise an input/output (I/O) device 130. The I/O device 130 may include a display device and an input device, which may respectively correspond to, for example, a computer video display and a hardware peripheral device (e.g., keyboard, mouse, etc.). In some embodiments, display device and input device may be combined or integrated into a single hardware device, such as a touchscreen. Generally, the I/O device 130 allows one or more users to interact with the functionality provided by modules 120, by one or both of (i) providing inputs to the modules 120, and (ii) perceiving outputs of the modules 120.

The forecasting computing system 102 may further comprise a network interface (NIC) 140 that allows the forecasting computing system 102 to send and receive network traffic (e.g., Internet Protocol packets) via the network 106. The forecasting computing system 102 may also comprise an electronic database for storing information related to the operation of the modules 120 and/or the particle processing and analysis system 104.

The particle processing and analysis system 104 comprises a suite of functionality for processing particles as discussed herein with respect to FIG. 2A and FIG. 2B. For example, the particle processing and analysis system 104 may include hardware and software for composing/preparing one or more formulations, for atomizing the one or more composed/prepared formulations, for performing spray drying and/or spray freeze drying, for generating powder blends, for performing spectroscopic analyses, etc. The particle processing and analysis system 104 may include a particle data collection module (not depicted) for collecting, formatting and transmitting data related to particle processing and analysis to the forecasting computing system 102 via the network 106.

It will be appreciated by those of ordinary skill in the art that in some embodiments, the topology of the computing environment 100 may be adjusted, or further simplified. For example, in an embodiment, the forecasting computing system 102 and the particle processing and analysis system 104 may correspond to the same physical server machine. In yet another embodiment, some or all of the functionality provided by the forecasting computing system 102 (e.g., the design generation module 122) may be implemented as an application accessible to users of the environment 100 in a mobile computing device (not depicted). In still further embodiments, a user of the particle processing and analysis system 104 may utilize a mobile computing device as an I/O device during an experiment facilitated by the module 124.

In operation, a user may prepare or select a composition and/or preparation of a formulation, for example by utilizing a particle processing method as described below. A user accesses the forecasting computing system 102 to configure at least one statistical experiment. For example, the user configures a Box-Behnken Design of Experiments (DoE) having three factors at three levels, to assess the stability of a protein (e.g., IgG1) under varying atomization conditions. The user may access input/output facilities of the forecasting computing system 102 in order to establish the statistical experiment. The user may select a design space that represents atomization conditions likely to be encountered in the particle engineering of respirable biopharmaceutical powders. For example, the user develops a design as follows:

| X1 coded | X2 coded | X3 coded | X1 uncoded | X2 uncoded | X3 uncoded |
|---|---|---|---|---|---|
| feed flow rate | atomization air | Solid content | Feed flow rate | Atomization air | Solid content |
| (mL/min) | flow rate (L/min) | (% w/v) | (mL/min) | flow rate (L/min) | (% w/v) |
| −1 | −1 | 0 | 1 | 13.9 | 5.5 |
| 1 | −1 | 0 | 3 | 13.9 | 5.5 |
| −1 | 1 | 0 | 1 | 22.9 | 5.5 |
| 1 | 1 | 0 | 3 | 22.9 | 5.5 |
| −1 | 0 | −1 | 1 | 18.4 | 1 |
| 1 | 0 | −1 | 3 | 18.4 | 1 |
| −1 | 0 | 1 | 1 | 18.4 | 10 |
| 1 | 0 | 1 | 3 | 18.4 | 10 |
| 0 | −1 | −1 | 2 | 13.9 | 1 |
| 0 | 1 | −1 | 2 | 22.9 | 1 |
| 0 | −1 | 1 | 2 | 13.9 | 10 |
| 0 | 1 | 1 | 2 | 22.9 | 10 |
| 0 | 0 | 0 | 2 | 18.4 | 5.5 |
| 0 | 0 | 0 | 2 | 18.4 | 5.5 |
| 0 | 0 | 0 | 2 | 18.4 | 5.5 |

Response factors assessed in the atomization DoE may include, for example, percent change in the amount of oligomer species, change in Z-average, change in secondary structure content, change in melting endotherm peak, and predicted median particle size. Each of the response factors may be measured using a stored particle analysis method, as described below.

The user accesses the design generation module 122 to set the design parameters as shown in the above table. In some embodiments, once the user has established the experimental parameters, and is ready to proceed, the user may select an indication in a graphical user interface of the forecasting computing system 102 that causes the stability assessment module 126 to generate and fit quadratic models to each response variable, to determine whether the quadratic models are predictive including, in some embodiments, the additional desirability function optimization to take place as discussed above. In some embodiments, the user may select another indication to cause the visualization module 128 to generate one or more visualizations corresponding to the predictive desirable models, advantageously assisting the user in understanding the significance of the generated models. It will be appreciated by those of ordinary skill in the art that in some case, little or no user input is required for the modules 120 to generate the predictive modeling. In other words, the process of setting up the statistical model/design parameters and performing the stability assessment may be partially, or fully, automated.

Once the predictive models are generated, the user may cause experiments to be initiated to test the atomization settings corresponding to the models, and the effect of those models on stability. For example, size exclusion chromatography and dynamic light scattering may be used to assess protein aggregation both before and after atomization.

The ability of the user to apply a statistical design, rather than searching for atomization conditions on an ad hoc basis advantageously results in more stable and accurate predictive models. By avoiding an exhaustive search, the present techniques may be used in cases where the amount of formulation is limited. Further avoiding exhaustive search advantageously saves time and significant resources. Still further, the present techniques allow the user to perform straightforward comparisons, and draw conclusions regarding, the behavior of different proteins in response to differing atomization conditions (e.g., IgG1-SD and IgG1-SFD).

Exemplary Particle Processing Method

Turning to FIG. 2A, an exemplary particle processing method 200 is depicted. By way of example, and not limitation, one or more steps of the particle processing method 200 may be performed by the experimentation module 124 of the forecasting computing system 102 of FIG. 1.

The particle processing method 200 may include composing/preparing one or more formulations (block 202). In some embodiments, a formulation may correspond to an immunoglobulin formulation (e.g., IgG1). For example, in some embodiments, a preferable IgG1 formulation having physicochemical properties most suitable for protein stability and aerosol performance may be a powder composition comprising 58.8% protein, 39.2% sucrose, 1.74% histidine, and 0.24% polysorbate 80.

Multiple composed/prepared formulations may correspond to different weight/volume concentrations. For example, a first formulation may correspond to a liquid feed formulation using the aforementioned IgG1 composition at 10% w/v. A second formulation may be prepared at 5.5% w/v, and a third formulation at 1% w/v. The preparation of formulations may include identifying a solid content level having the most optimal balance of high protein stability and small predicted geometric particle size distribution (PSD). The final pH of the formulations may be, for example, 6.3. It should be appreciated that the foregoing example is simplified for explanatory purposes, and the formulation may include any suitable composition, prepared at any suitable weight/volume concentration, and having a suitable pH.

The particle processing method 200 may include atomizing the one or more composed/prepared formulations at block 202 (block 204). For example, the particle processing method 202 may include atomization of IgG1 using a two-fluid pneumatic nozzle with 0.7 mm nozzle and 1.5 mm nozzle cap, wherein the cleaning needle is removed from the nozzle to prevent the generation of negative pressure from disrupting the flow rate. The atomizing at block 204 may include using compressed nitrogen as the atomization gas. The atomization air flow rate may be set using a flow meter positioned at the nozzle exit. The feed flow rate may be set using a syringe pump. In some embodiments, atomizing may include atomizing formulations into a 50 mL glass vial set at a fixed distance below the nozzle.

The particle processing method 200 may include performing spray drying and/or spray freeze drying to generate one or more respective powders (block 206). For example, in an embodiment, the particle processing method 200 may perform spray drying using a BUCHI B-290 spray dryer with de-humidifier attachment, having an aspiration rate set at 100% and inlet temperature set at 130° C.

In an embodiment, the particle processing method 200 may perform spray freezing into liquid nitrogen to generate a formulation slurry. Once frozen, the particle processing method 200 may include pouring the liquid nitrogen formulation slurry was into one or more glass vials, loosely covered and loaded into a pre-chilled shelf lyophilizer (e.g., a VirTis BenchTop lyophilizer). In some embodiments, the particle processing method 200 performs lyophilization according to published method familiar to those of ordinary skill in the art.

In some embodiments, the particle processing method 200 may include generating an experimental control with respect to one or both of the spray drying and spray freeze drying processes by lyophilization. For example, to continue the example, in some embodiments the particle processing method 200 may lyophilize the IgG1 formulation (e.g., IgG1-Lyo) using identical settings to the spray freeze drying process. After the secondary drying step, the particle processing method 200 may include filling the lyophilized chamber with nitrogen gas, completely closing the vials (e.g., using rubber stoppers) using compressed air inside the chamber, and sealing the vials with aluminum caps.

In some embodiments, the particle processing method 200 may include generating powder blends to assess long-term effects on crystallinity and protein stability. Continuing the example, the particle processing method 200 may include generating IgG1-lactose powder blends using the prepared spray dried and spray freeze dried powders and a dry powder inhalation excipient (e.g., Lactohale 100 lactose monohydrate). The particle processing method 200 may include blending resulting formulations at 10% w/w with lactose (equivalent to ˜5-6% IgG1) and combining the powders using a process of geometric dilution then mixed (e.g., using a Turbula Mixer) for a time (e.g., 2 hours), followed by grinding (e.g., in a mortar and pestle) until percentage coefficient of variation (% CV) of less than 5% was achieved for IgG1.

The particle processing method 200 may include storing the one or more respective powders (block 208). For example, continuing the example, the particle processing method 200 may include storing the IgG1 powders for 30 days at 25° C./60% RH and 40° C./75% RH, as specified by the International Council For Harmonisation (ICH) intermediate and accelerated stability conditions. For example, the particle processing method 200 may include filling IgG1-SD, IgG1-SFD-lac, and IgG1-SD-lac into size capsules (e.g., #3 HPMC inhalation-grade capsules) at a target weight of 20 mg, and IgG1-SFD at a target weight of 5 mg based upon the lower density of the powder. Following encapsulation, the particle processing method 200 may include placing the capsules in the lyophilizer for 8 hours at 100 mTorr to remove water adsorbed during the encapsulation process and placing the capsules in HDPE vials sealed in foil bags without desiccant. The particle processing method 200 may include storing the IgG1-Lyo powder in rubber-stopped vials inside foil bags with desiccant. The method may include storing particle processing method 200 bulk Ig1-SFD and IgG1-SD powders in a similar manner to IgG1-Lyo as a further control.

Exemplary Stored Particle Processing Method

FIG. 2B depicts a stored particle analysis method 250, according to an embodiment. The stored particle analysis method 250 may include performing physicochemical characterization of the powders, examination of structural integrity (e.g., IgG1 structural integrity), and/or aerosol performance analysis in comparison to baseline metrics after a suitable time (e.g., after 30 days). By way of example and not limitation, the stored particle analysis method 250 may be performed by the stability assessment module 126 of the forecasting computing system 102 of FIG. 1.

The stored particle analysis method 250 may include analyzing structure and/or aggregation of the powders stored in block 208 of FIG. 2 (block 252). For example, the particle analysis method 250 may examine unprocessed and processed mAb (e.g., IgG1) for changes in aggregation, secondary structure, and tertiary structure. The stored particle analysis method 250 may use size exclusion chromatography (SEC-HPLC) to quantify presence of soluble aggregates using previously published methods that will be familiar to those of ordinary skill in the art. The stored particle analysis method 250 may determine oligomer types present in the samples by referencing a curve of SEC using proteins of varying molecular weights. In some embodiments, the stored particle analysis method 250 may use dynamic light scattering as an alternative/complementary technique to SEC. In some embodiments, a Malvern Zetasizer may be used to automatically find the most optimal settings for the sample, using a number of size measurements (e.g., three) per sample.

The stored particle analysis method 250 may detect the presence of insoluble aggregates at block 252. For example, the stored particle analysis method 250 may measure turbidity of the IgG1 powders dissolved in 100 mM sodium phosphate and 250 mM sodium chloride buffer at a wavelength of 340 nm-360 nm and 690 nm, wherein the powders are dissolved to a given concentration (e.g., 0.6 mg/mL protein).

For the atomization experiments, the stored particle analysis method 250 may use circular dichroism (CD) spectroscopy to assess changes in protein secondary structure at block 252 in the liquid state.

The stored particle analysis method 250 may include using differential scanning calorimetry (DSC) to assess changes in protein tertiary structure at block 252. The start and midpoint of the endothermic peak may be determined using software. Changes in protein tertiary structure also may be monitored by observing the fluorescence emission maxima of samples.

In some embodiments, the stored particle analysis method 250 may include assessing a particle size distribution (PSD) of stored powders (e.g., IgG1-SD and IgG1-SFD) at baseline and after storage using a laser diffraction unit coupled with a dry powder disperser (e.g., RODOS) (block 254). The stored particle analysis method 250 may determine the effect of storage on powder dispersibility by taking PSD measurements at a plurality of bar dispersion pressures (e.g., 0.2 bar and 4 bar). The stored particle analysis method 250 may compute a final PSD by averaging time slices in each plume corresponding to an optical concentration between 5% and 25%.

The stored particle analysis method 250 may include examining morphology of powder (e.g., IgG1 powder) at baseline using a scanning electron microscope (SEM) such as a Zeiss Supra 40VP SEM, for example. The stored particle analysis method 250 may include mounting samples onto aluminum stubs using double-side carbon tape and sputter coated with 15 nm of platinum/palladium (Pt/Pd) under argon using a sputter coater (e.g., a Cressington sputter coater 208 HR).

The stored particle analysis method 250 may include performing x-ray powder diffraction (XRD) analysis to determine storage induced changes in powder crystallinity (block 256). For example, the stored particle analysis method 250 may include scanning samples in continuous mode from 5-45° C. at a rate of 1°/min and a step size of 0.025° using a Riguku MiniFlex II equipped with a radiation source generated at 40 kV and 15 mA. The stored particle analysis method 250 may include measuring changes in glass transition temperature using modulated DSC.

The stored particle analysis method 250 may include assessing the aerosol performance of powders (e.g., IgG1) at baseline and after storage using a cascade impactor (e.g., a Next Generation Impactor) and a high resistance DPI (e.g., an RS01 Monodose DPI) (block 258).

The stored particle analysis method 250 may include determining differences between powders produced using two or more of lyophilization, spray drying or spray freeze drying at baseline and after a period (e.g., 30 days) of storage at intermediate and/or accelerated stability conditions for statistical significance (block 260). For example, the stored particle analysis method 250 may include calculating analysis of variance (ANOVA) with Tukey post hoc analysis, and alpha of 0.05.

Generally, the present techniques may include using one or more steps of the particle processing method 200 and/or one or more steps of the stored particle analysis method 250 to analyze atomization conditions/parameters identified by the design generation module 122 as being optimal. For example, in an embodiment, one or more respective steps of the particle processing method 200 and the stored particle analysis method 250 may be used to assess the effect of spray drying and spray freeze drying on structure and aggregation of IgG1.

Exemplary Response Surface Visualizations

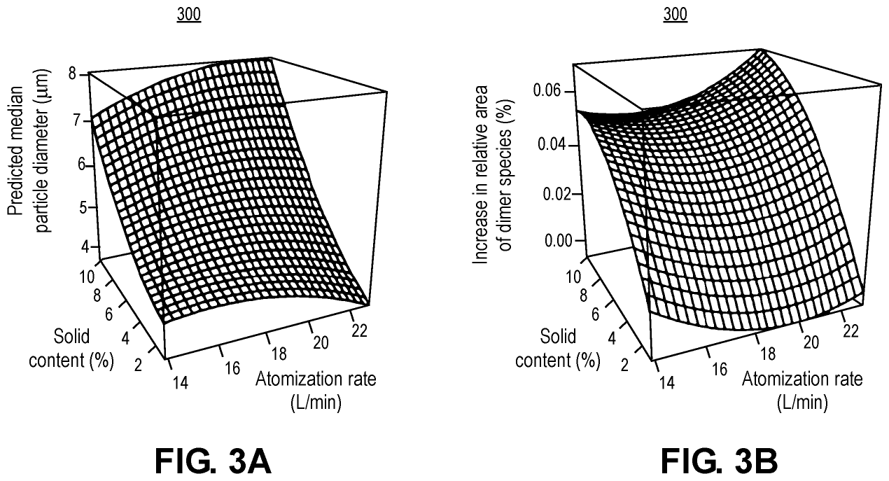

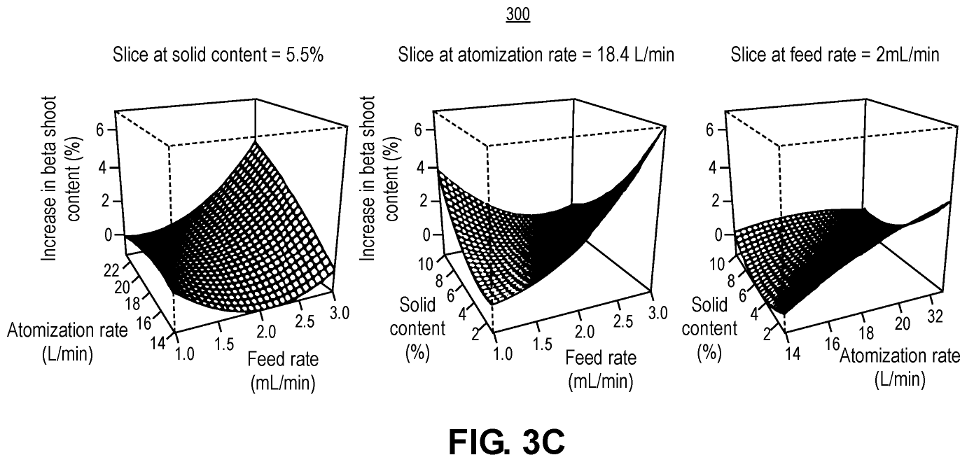

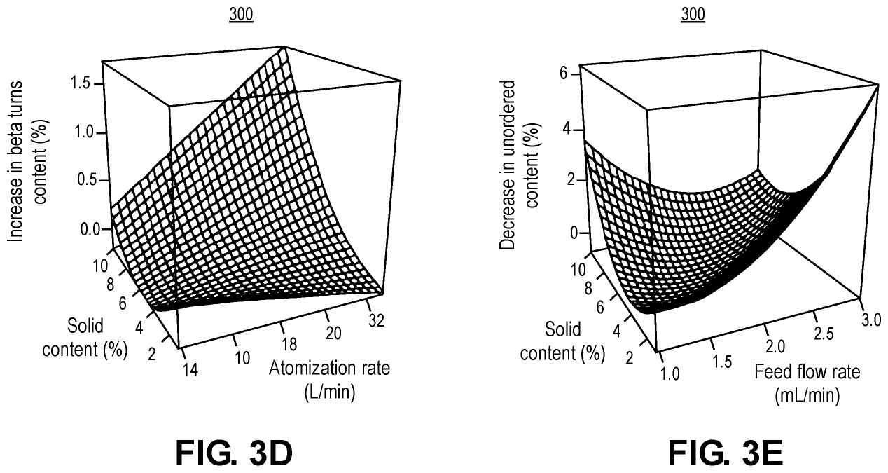

FIGS. 3A-3E depicts exemplary response surface visualizations corresponding to predictive quadratic equations describing desirable atomization settings. As discussed above, the present techniques do not rely on guessing or interpolation to determine optimal settings.

Rather, the surface response visualizations as shown in FIGS. 3A-3E may assist a user in finding optimal values. It follows, then, that even if the user is analyzing a protein about which the user knows nothing, the visualizations as shown in FIGS. 3A-3E can assist the user in immediately determining that a protein is more or less easy to stabilize, by how sensitive it is to process parameters. As such, the visualization capabilities of the present techniques advantageously provide the researcher with quick and intuitive clues regarding the likely behavior of novel formulations. This may assist the user in avoiding selection of formulations that are likely to slow down a project or not work, or to select a more suitable formulation, thus saving energy and resources.

Those of ordinary skill in the art will appreciate that many formulation attributes can cause a formulation to be unsuitable, such as too much or too little viscosity, inadequate surface tension, excess or deficient protein loading, etc. The response surface visualizations may allow users to avoid costly mistakes by providing a visual guide to such unsuitable compounds/formulations.

Exemplary Method for Determining Optimal Atomization Settings

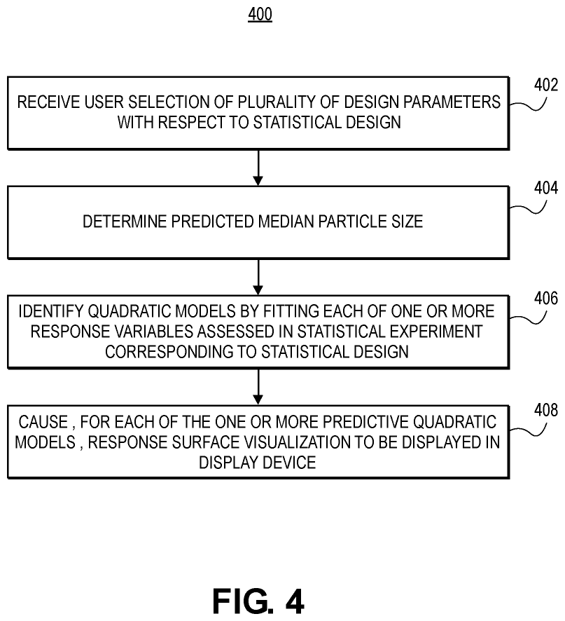

Turning to FIG. 4, an exemplary method 400 for determining optimal formulation atomization settings in a particle engineering process of a protein is depicted.

The method 400 may include receiving, in a design generation module of a forecasting computing system, a user selection of a plurality of design parameters with respect to a statistical design (block 402). For example, the design generation module of the forecasting computing system may correspond to the design generation module 122 of the forecasting computing system 102 of FIG. 1. The user selection may be facilitated by, for example, a graphical user interface displayed in the I/O device 130 of FIG. 1. The user selection may include one or more selections defining a statistical design such as a Box-Behnken DoE experiment, including parameters. The parameters, or design space, may be selected to represent atomization conditions typically used in particle engineering of respirable biopharmaceutical powders, in some embodiments. When the statistical design is a Box-Behnken Design of Experiment, the assessed response variables may include one or more of a percent change in the amount of oligomer species, a change in Z-average, a change in secondary structure content, a change in melting endotherm peak, or a predicted median particle size.

The method 400 may include determining, in a suitability assessment module of the forecasting computing system, a predicted median particle size (block 404). The suitability assessment module of the forecasting computing system may correspond to the suitability assessment module 126 of the forecasting computing system 102. The predicted median particle size may be determined using an equation as discussed above. The predicted median particle size is determined by analyzing a respective droplet size, a weight fraction, and a dried particle size. Specifically, the resulting droplet size for each atomization setting may be determined using laser diffraction, and used to calculate a predicted median particle size according to:

$${D}_{d} = \frac{{D}_{w}}{{( \frac{{ρ}_{D}}{{ρ}_{w}} \cdot \frac{{C}_{D}}{{C}_{w}} )}^{\frac{1}{3}}}$$

The method 400 may include identifying one or more predictive quadratic models by fitting each of one or more response variables assessed in a statistical experiment corresponding to the statistical design (block 406). For example, quadratic equations may be fit to predicted median particle size, increase in SEC-HPLC dimer relative peak area versus average unprocessed control, etc. Those fits wherein F-test P value and lack of fit test P value are respectively significant and insignificant result in an identification of a given model being predictive. In some embodiments, the forecasting computing system 102 may access experimental data via the experimental module 124 and/or the particle processing and analytics system 104, for example to compare processed and unprocessed protein, or to retrieve stored particle data.

In some cases, the method 400 may include applying a desirability function to further optimize the predictive nature of quadratic models deemed predictive at block 406. As noted above, the equation used to determine the most suitable atomization conditions is:

$$d \frac{min}{r} \{ \begin{matrix}0 & if {f}_{r} ( x ) > B \\ ( \frac{{f}_{r} ( x ) - {B}^{s}}{A - B} ) & if A \leq {f}_{r} ( x ) \leq B \\ 1 & {f}_{r} ( x ) < A\end{matrix}$$

The method 400 may include causing, in a visualization module of the forecasting computing system, for each of the one or more predictive quadratic models, a response surface visualization to be displayed in a display device of a user (block 408). For example, several predictive quadratic equations are depicted in FIGS. 3A-3D. The visualization module 128 of the forecasting computing system 102 may cause such visualizations to be generated and/or displayed in the I/O device 130 of the forecasting computing system 102, in a display device of a remote web client, in a mobile device of a user, etc. In some embodiments, the visualization module 128 may transmit generated visualizations (e.g., to the database 150 for storage, via email, etc.).

Generally, the proteins acted upon by the method 400 may be any suitable proteins, including without limitation, antibodies (e.g., monoclonal antibodies). Generally, particle engineering process of the method 400 may be configured to create a respirable biopharmaceutical powder, and may be configured to deliver the respirable biopharmaceutical powder via one or both of (i) a nebulizer, and (ii) a dry powder inhaler.

In some embodiments, the method 400 may include causing, in a particle processing and analysis system such as the particle processing and analysis system 104 of FIG. 1, a subsequent experiment to be initiated using the predictive quadratic models to control atomization settings. For example, a subsequent experiment may vary one or more atomization settings (e.g. based on the output of the desirability function). The method 400 may compare a result of the subsequent experiment to a result of the statistical experiment corresponding to the statistical design.

In some embodiments, the method 400 may include receiving, from a particle processing and analysis system, experimental data corresponding generated by a particle processing method, the experimental data corresponding to the one or more assessed response variables. For example, the particle processing and analysis system 104 may automatically transmit data describing flow rate of a flow meter to the forecasting computing system. The method 400 may further include receiving, from a particle processing and analysis system, experimental data corresponding generated by a stored particle analysis method, the experimental data corresponding to the one or more assessed response variables. For example, data describing circular dichroism spectroscopy used to assess changes in secondary structure of the protein may be received/retrieved by the forecasting computing system 102 of FIG. 1 and/or transmitted by the particle processing and analysis system 104 of FIG. 1.

Additional Considerations

All of the references cited herein, including patents, patent applications, literature publications, and the like, are hereby incorporated in their entireties by reference.

It should also be understood that when describing a range of values, the disclosure contemplates individual values found within the range. For example, “cell aggregate size of between about 20 nm and about 200 nm,” could be, but is not limited to, 40 nm, 60 nm, 100 nm, etc., and any value in between such values. In any of the ranges described herein, the endpoints of the range are included in the range. However, the description also contemplates the same ranges in which the lower and/or the higher endpoint is excluded.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based upon the application of 35 U.S.C. § 112(f). The systems and methods described herein are directed to an improvement to computer functionality, and improve the functioning of conventional computers.

Throughout this specification, the word “set”, unless expressly defined otherwise, is hereby defined to mean a set having one or more elements, but not the empty set.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules can provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may then, at a later time, access the memory device to retrieve and process the stored output. Modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment. In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

This detailed description is to be construed as exemplary only and does not describe every possible embodiment, as describing every possible embodiment would be impractical, if not impossible. One could implement numerous alternate embodiments, using either current technology or technology developed after the filing date of this application. Upon reading this disclosure, those of ordinary skill in the art will appreciate still additional alternative structural and functional designs for performing the system and method of the present disclosure through the disclosed principles herein. Thus, while particular embodiments and applications have been illustrated and described, it is to be understood that the disclosed embodiments are not limited to the precise construction and components disclosed herein. Various modifications, changes and variations, which will be apparent to those of ordinary skill in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein. It is therefore intended that the foregoing detailed description be regarded as illustrative rather than limiting, and that it be understood that it is the following claims, including all equivalents, that are intended to define the spirit and scope of this invention.

1. A forecasting modeling computing system for optimizing atomization settings during particle engineering of a protein, comprising:
one or more processors; and
a memory including a set of computer-executable instructions that, when executed by the one or more processors, cause the forecasting modeling computing system to:
receive, in a design generation module of the memory, a user selection of a plurality of design parameters with respect to a statistical design,
determine, in a suitability assessment module of the memory, a predicted median particle size,
identify, in a stability assessment module of the memory, one or more predictive quadratic models by fitting each of one or more response variables assessed in a statistical experiment corresponding to the statistical design; and
cause, in a visualization module of the memory, for each of the one or more predictive quadratic models, a response surface visualization to be displayed in a display device of a user.

2. The forecasting modeling computing system of claim 1, the memory including further instructions that, when executed, cause the forecasting modeling computing system to:
applying a desirability function to further optimize the predictive quadratic models.

3. The forecasting modeling computing system of either claim 1, wherein the protein is an antibody.

4. The forecasting modeling computing system of claim 1, wherein the predictive modeling computing system is used to make a respirable biopharmaceutical powder.

5. The forecasting modeling computing system of claim 4, the memory including further instructions that, when executed, cause the predictive modeling computing system to:
deliver the respirable biopharmaceutical powder via one or both of (i) a nebulizer, and (ii) a dry powder inhaler.

6. The forecasting modeling computing system of claim 1, wherein the assessed response variables include one or more of:
a percent change in the amount of oligomer species,
a change in Z-average,
a change in secondary structure content,
a change in melting endotherm peak, or
a predicted median particle size.

7. The forecasting modeling computing system of claim 1, wherein the predicted median particle size is determined by analyzing a respective droplet size, a weight fraction, and a dried particle size.

8. The forecasting modeling computing system of claim 1, the memory including further instructions that, when executed, cause the predictive modeling computing system to:
cause, in a particle processing and analysis system, a subsequent experiment to be initiated using the predictive quadratic models to control atomization settings; and
compare a result of the subsequent experiment to a result of the statistical experiment corresponding to the statistical design.

9. The forecasting modeling computing system of claim 1, the memory including further instructions that, when executed, cause the predictive modeling computing system to:
receive, from a particle processing and analysis system, experimental data corresponding generated by a particle processing method, the experimental data corresponding to the one or more assessed response variables.

10. The forecasting modeling computing system of claim 1, the memory including further instructions that, when executed, cause the predictive modeling computing system to:
receive, from a particle processing and analysis system, experimental data corresponding generated by a stored particle analysis method, the experimental data corresponding to the one or more assessed response variables.

11. A computer-implemented method for determining optimal formulation atomization settings in a particle engineering process of a protein, comprising:
receiving, in a design generation module of a forecasting modeling computing system, a user selection of a plurality of design parameters with respect to a statistical design,
determining, in a suitability assessment module of the forecasting modeling computing system, a predicted median particle size,
identifying one or more predictive quadratic models by fitting each of one or more response variables assessed in a statistical experiment corresponding to the statistical design; and
causing, in a visualization module of the forecasting modeling computing system, for each of the one or more predictive quadratic models, a response surface visualization to be displayed in a display device of a user.

12. The computer-implemented method of claim 11, further comprising:
applying a desirability function to further optimize the predictive quadratic models.

13. The computer-implemented method of either claim 11, wherein the protein is an antibody.

14. The computer-implemented method of claim 11, wherein the particle engineering process is configured to create a respirable biopharmaceutical powder.

15. The computer-implemented method of claim 14, further comprising:
delivering the respirable biopharmaceutical powder via one or both of (i) a nebulizer, and (ii) a dry powder inhaler.

16. The computer-implemented method of claim 11, wherein the statistical design is a Box-Behnken Design of Experiment and the assessed response variables include one or more of:
a percent change in the amount of oligomer species,
a change in Z-average,
a change in secondary structure content,
a change in melting endotherm peak, or
a predicted median particle size.

17. The computer-implemented method of claim 11, wherein the predicted median particle size is determined by analyzing a respective droplet size, a weight fraction, and a dried particle size.

18. The computer-implemented method of claim 11, further comprising:
cause, in a particle processing and analysis system, a subsequent experiment to be initiated using the predictive quadratic models to control atomization settings; and
comparing a result of the subsequent experiment to a result of the statistical experiment corresponding to the statistical design.

19. The computer-implemented method of claim 11, further comprising:
receiving, from a particle processing and analysis system, experimental data corresponding generated by a particle processing method, the experimental data corresponding to the one or more assessed response variables.

20. The computer-implemented method of claim 11, further comprising:
receiving, from a particle processing and analysis system, experimental data corresponding generated by a stored particle analysis method, the experimental data corresponding to the one or more assessed response variables.
